(19) United States
(12) Patent Application Publication
(10) Pub. No.: US 2009/0112311 A1
(43) Pub. Date: Apr. 30, 2009

(54) MEDICAL DEVICE FOR PERCUTANEOUS PARAVALVULAR LEAK AND RELATED SYSTEMS AND METHODS

(75) Inventors: Scott D. Miles, Sandy, UT (US); Clark C. Davis, Holladay, UT (US); Richard J. Linder, Sandy, UT (US)

Correspondence Address: Holland & Hart LLP/Coherex Medical, 60 East South Temple, Suite 2000, Salt Lake City, UT, 84111, US

(73) Assignee: COHEREX MEDICAL, INC., Salt Lake City, UT (US)

(21) Appl. No.: 12/253,882

(22) Filed: Oct. 17, 2008

Related U.S. Application Data
(60) Provisional application No. 60/981,454, filed on Oct. 19, 2007.

(51) Int. Cl. A61F 2/24

(52) U.S. Cl. 623/2.38

(57) ABSTRACT

The present invention provides devices, systems and methods for closing a paravalvular leak. In accordance with one embodiment, a medical device includes at least one multicellular frame member configured to be implanted at a paravalvular leak. The medical device further includes at least one tissue in-growth member associated with the frame member, the tissue in-growth member being configured to promote tissue growth and permanently maintain the frame member at the leak. The frame member may be self expanding device formed, for example, of a shape-memory alloy. The tissue in-growth member may be formed from a polymer material. In one particular embodiment, the frame member may be a substantially tubular structure and the tissue in-growth member may be disposed within an interior space defined by the tubular structure. In another embodiment, the frame ember may be a substantially flat or planar structure.

### CROSS-REFERENCE TO RELATED APPLICATION

This application claims the benefit of Unites States Provisional Patent Application Ser. No. 60/981,454, filed Oct. 19, 2007, entitled FOR PERCUTANEOUS PARAVALVULAR LEAK, the disclosure of which is incorporated by reference herein in its entirety.

### TECHNICAL FIELD

The present invention relates generally to medical devices and, more particularly, to medical devices that may be delivered percutaneously for repair of paravalvular leaks.

### BACKGROUND

A relatively common mode of heart-failure is for the valves of the heart to fail functionally. This may happen due to age, disease or many other factors, but the result is a reduction in the pumping efficiency of the heart which increases the amount of stress that the heart experiences in order to move the same amount of blood. In other words, the heart becomes less efficient and needs to work harder to maintain a healthy flow of blood.

When one's heart valves begin to fail, they may, for example, be replaced with artificial valves. These valves may be mechanical, constructed of biological tissues, or they may be cadaveric valves from other animal species. In all of these replacement valves, an important feature of their implantation is their fixation in the correct position within the heart. This is typically done with what is referred to as a “sewing ring.” A sewing ring is an annular component configured to surround the outer periphery of the implant that may be sutured into place in the heart. In cases where the ring or (other attachment device or system) is not entirely incorporated into the heart around its entire periphery, leaks may occur between the natural heart tissue and the implant.

In recent years, valve implants which are delivered percutaneously have been introduced. In these valve implants, leaks may be more common than those implanted surgically. Some devices have been developed in an effort to reduce leaks when incorporated with a valve replacement. For example, U.S. Pat. No. 7,276,078 to Spenser et al. discloses a percutaneous device for the repair of heart valves including “means for leak prevention.”

However, it remains desirable to be able to repair paravalvular leaks or failures, regardless of the manner in which a valve replacement or repair has been conducted. It also remains desirable to effect such leak repairs in a manner that is relatively non-invasive. For example, it may be desirable to effect such repairs through a percutaneous procedure whether or not the artificial valve was replaced percutaneously or through a more invasive surgical procedure.

### BRIEF SUMMARY

Embodiments of the present invention relate to medical devices that may be used to repair valvular leaks with an additional, percutaneously delivered implant. In particular, embodiments of the present invention relate to medical devices for repairing a paravalvular leak.

In accordance with one embodiment of the present invention, a medical device for repairing a paravalvular leak is provided. The medical device includes at least one multicellular frame member configured to be implanted at a paravalvular leak. The medical device further includes at least one tissue in-growth member associated with the frame member, the tissue in-growth member being configured to promote tissue growth and permanently maintain the frame member at the leak. The frame member may be self expanding device formed, for example, of a shape-memory alloy. The tissue in-growth member may be formed from a polymer material. In one particular embodiment, the frame member may be a substantially tubular structure and the tissue in-growth member may be disposed within an interior space defined by the tubular structure. In another embodiment, the frame member may be a substantially flat or planar structure.

In accordance with another embodiment of the present invention, a method is provided for closing a paravalvular leak. The method includes disposing a multicellular frame member within the paravalvular leak and disposing a tissue in-growth member within the paravalvular leak at a location adjacent the multicellular frame member to promote tissue growth and permanently maintain the frame member at the leak. In one embodiment, disposing the frame member within the paravalvular leak and disposing the tissue in-growth member within the paravalvular leak may occur substantially simultaneously.

In accordance with yet another embodiment of the present invention, another medical device is provided. The medical device includes a frame member having at least two elongated arms biased in two different directions. A tissue in-growth member configured to be attached to the frame member to promote tissue growth and permanently seal the leak. In one embodiment, the two different direction in which the at least two elongated arms are biased are substantially opposite one another. In another embodiment, the at least two elongated arms include at least two pair of elongated arms. Tines may be associated with the elongated arms to engage walls surrounding the paravalvular leak and prevent migration of the medical device.

These and other aspects of the present invention will become more fully apparent from the following description and appended claims, or may be learned by the practice of the invention as set forth hereinafter.

### BRIEF DESCRIPTION OF THE DRAWINGS

To further clarify the above and other aspects of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The drawings are not drawn to scale. The invention will be described and explained with additional specificity and detail through the use of the accompanying drawings in which:

FIG. 1 shows an artificial valve incorporated into a heart and depicting a valvular leak location;

FIGS. 2A through 2C show end, side and perspective views of a medical device according to an embodiment of the present invention;

FIG. 3 shows an end view of a medical device according to another embodiment of the present invention;

FIGS. 4A and 4B show an end view and a side view of a medical device according to an embodiment of the present invention;

FIG. 5 shows a medical device deployed to remedy a paravalvular leak according to an embodiment of the present invention;

FIG. 6 shows a side view of a medical device according to an embodiment of the present invention;

FIG. 7 shows a perspective view of a medical device according to an embodiment of the present invention;

FIG. 8 shows a medical device deployed to remedy a paravalvular leak according to an embodiment of the present invention;

FIG. 9 shows a medical device according to another embodiment of the present invention;

FIG. 10 shows the medical device of FIG. 8 deployed to remedy a paravalvular leak according to an embodiment of the present invention; and

FIGS. 11A and 11B show side and end views of a medical device according to another embodiment of the present invention.

### DETAILED DESCRIPTION OF THE INVENTION

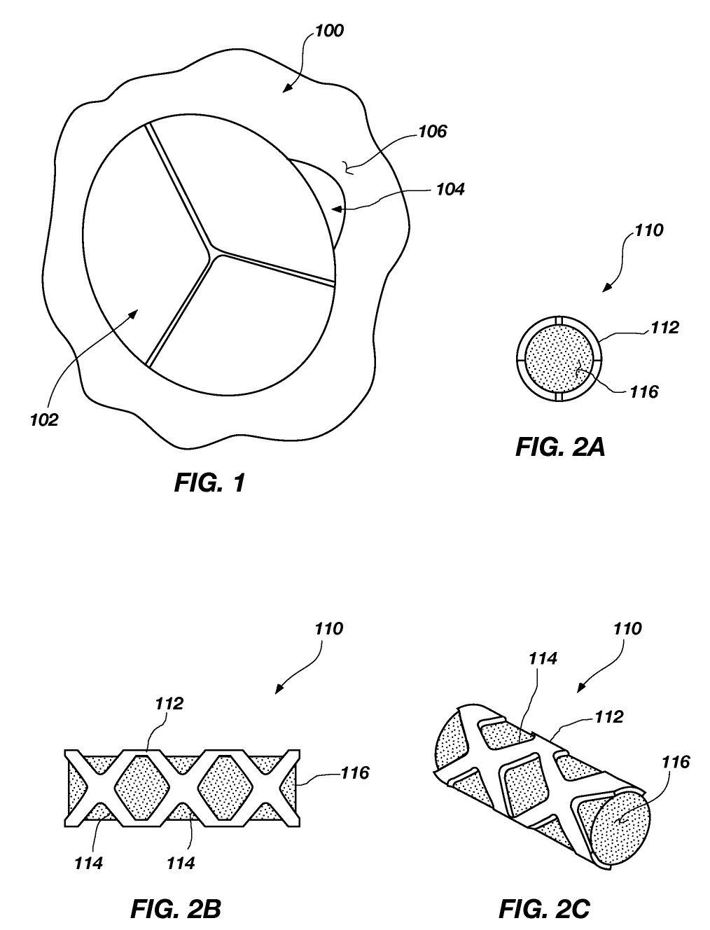

FIG. 1 illustrates an artificial valve 100 that may include, for example, a multi-leaflet structure 102 and that may be implemented in an appropriate manner as will be appreciated by those of ordinary skill in the art. Additionally, a small opening or space 104 between the heart 106 and the valve 100 is shown as may occur in some instances of valve replacement as has been described hereinabove. This opening or space 104 results in undesired leaking during pumping of the heart and is termed a valvular or paravalvular leak. Various embodiments of the present invention are described herein for blocking, plugging or otherwise occluding the space 104 and reducing or eliminating any leaks therethrough.

FIGS. 2A-2C illustrates an embodiment of a medical device 110 that is sized and configured to be positioned at a valvular leak (e.g., within space 104) and to block the unwanted flow of blood therethrough. The medical device 110 may include a tubular, multicellular frame member 112 formed of a plurality of struts 114. It is noted that the term tubular is not intended to indicate a continuous tube in the strict sense but, rather, is intended to describe a generally elongated structure having a defined passageway within and along the length of the elongated structure. A tissue in-growth member 116 may be disposed within, or otherwise associated with, the tubular frame member 112. Such in-growth member 116 may be formed of any suitable in-growth member known to one of ordinary skill in the art. For example, in one embodiment, the in-growth material may include a polymer material such as a polymer based fabric, a reticulated polymer foam, silicone, polyurethane, polyvinyl acetate, or polymer or metallic felt.

In one embodiment, the frame member 112 may be formed as a stent-like structure configured to be displaced between a contracted state for deliver thereof to the site of a leak, and an expanded state for operational or functional purposes. The frame member 112 may be deployed by way of a catheter, similar to stent structures, as will be appreciated by those of ordinary skill in the art. The tissue in-growth member 116 may then be disposed within the expanded frame member 112 after the frame member 112 has been deployed in the space 104. The in-growth member 116 may be delivered by a catheter, similar to the frame member 112, except that the tissue in-growth member 114 may be self expanding when released from a catheter or other delivery mechanism. When disposed within the opening or space 104, the frame structure 112 and tissue in-growth member 116 serve to block the flow path through the space 104. Additionally, the tissue in-growth member 116 helps to promote or advance tissue growth and help permanently fix the medical device 110 within the space 104.

In another embodiment, the medical device 110 may include a self expanding frame member 112 made, for example, of a shape-memory alloy (e.g., a nickel titanium alloy such as Nitinol) having the tissue in-growth member 116 already disposed within the frame member 112. Such an embodiment enables the medical device 110 to be delivered in a single act (i.e., the frame member 112 and tissue in-growth member 116 may be delivered effectively simultaneously as compared to the embodiment previously described wherein the in-growth member 116 is disposed within the frame member 112 after expansion of the frame member 112) and, thus, simplifies the deployment process and ease of use of the medical device 110.

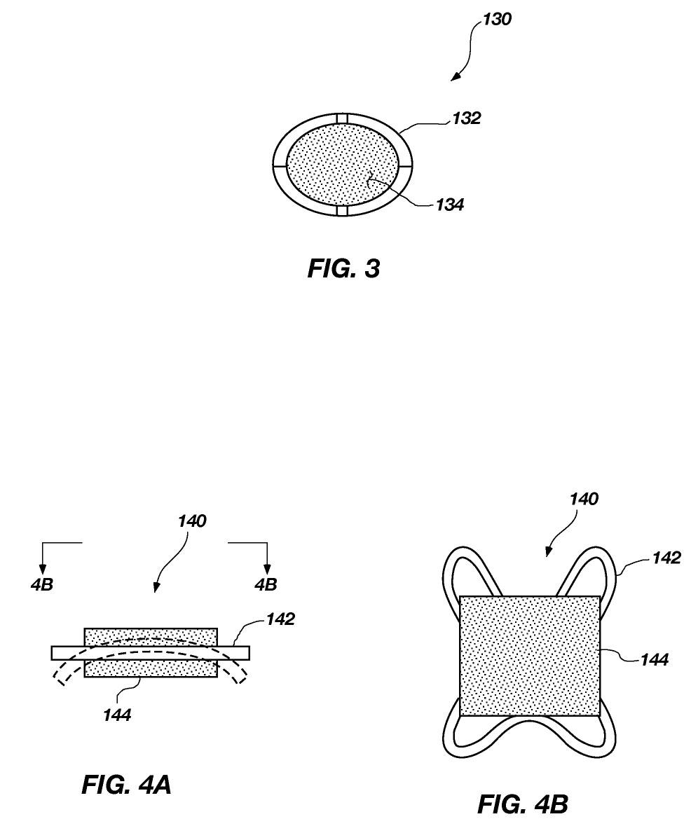

Referring to FIG. 3 another embodiment of a medical device 130 is shown and described. The medical device 130 is generally similar to the previously described embodiment having a generally tubular frame member 132 and the tissue in-growth material 134 positioned within the frame member 132. The frame member 132 exhibits a generally oval cross-sectional periphery as compared to the substantially circular periphery of the medical device 110 described with respect to FIGS. 2A-2C. Other profiles, shapes and geometries may also be utilized so long as the provide appropriate structural support by way of the frame member 132 while also facilitating tissue growth with the tissue in-growth material 134, thereby, forming a permanent seal for the valvular leak.

FIGS. 4A-4B illustrate another embodiment for a medical device 140 that can provide a permanent seal for a valvular leak. In this embodiment, the medical device 140 includes a frame member 142 having a low profile as compared to previously described embodiments. The low profile frame member 142 may be substantially flat or planar and may be bendable (such that it exhibits a curved or arcuate profile as shown in dashed lines in FIG. 4A) to facilitate conformal placement within the space that defines the valvular leak. The medical device 140 may also include the tissue in-growth material 144, which may include, for example, a polymer substrate such as a foam, felt or a fabric material as has been described hereinabove. A more detailed example of a planar or flat frame member is set forth in U.S. patent application Ser. No. 11/836,123 filed on Aug. 8, 2007, entitled METHODS, SYSTEMS, AND DEVICES FOR REDUCING THE SIZE OF AN INTERNAL TISSUE OPENING and assigned to the assignee hereof, the disclosure of which is incorporated by reference herein in its entirety.

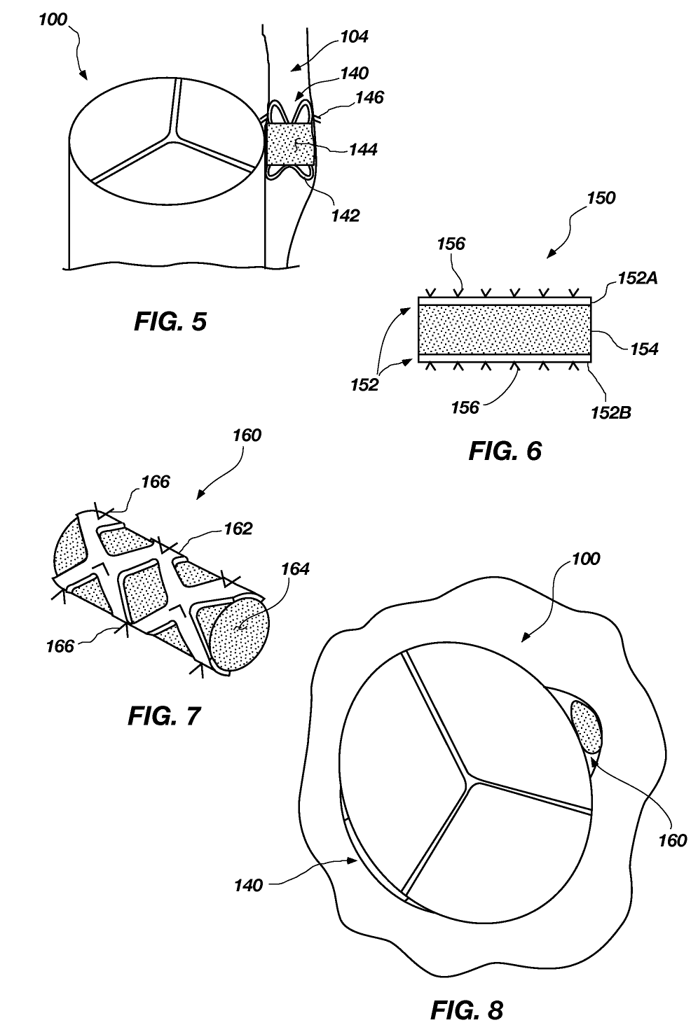

As in the previously described embodiments, the tissue in-growth material 144 acts as a plug for the valvular leak as well as a means for promoting tissue growth and, thus, a permanent seal for the valvular leak. In presently described embodiment (i.e., with respect to FIGS. 4A and 4B), the low profile nature of the frame member 142 may provide the advantage of being more readily implantable within certain valvular leaks. For example, as shown in FIG. 5, the medical device 140 is disposed within a valvular leak path. While not shown in FIGS. 4A and 4B, the frame member 142 may optionally include tines 146 oriented so as to prevent the medical device 140 from migrating out of the space 104 that defines the valvular leak.

FIG. 6 illustrates another embodiment of a medical device 150 for blocking a valvular leak. The medical device 150 may include a frame structure 152 having two primary frame members 152A and 152B each having a low profile. In other words, each of the frame members 152A and 152B may be generally similar to the frame member 142 described with respect to FIGS. 5A and 5B. A tissue in-growth member 154 positioned between the two frame members 152A and 152B. Each frame member 152A and 152B may be substantially flat or planar and may be positioned relatively parallel to one another. The two frame members 152A and 152B may be interconnected to each other via the tissue in-growth member 154, they may be interconnected via strut members (not shown) or cross-members, or by both means. Such frame members 152A and 152B may be sized and configured to facilitate slight bending to a curved or arcuate shape (such as has been described above) so as to enable the medical device 150 to fit within a valvular leak having a slight curved profile. As in the previous embodiment, the frame members 152A and 152B may also include tines 156 to prevent migration of the medical device 150.

Referring briefly to FIG. 7, another embodiment of a medical device 160 is shown. The medical device 160 is generally similar to the medical device 110 described with respect to FIGS. 2A-2C in that it includes a generally tubular frame member 162 and a tissue in-growth member 164. The medical device 160 further includes a plurality of tines 166 extending from the outer periphery of the frame member 162 to engage surrounding tissue or structure and prevent migration of the medical device 160 out of the space defining the valvular leak.

FIG. 8 illustrates a perspective view of an artificial valve 100 with two different leak paths being plugged or blocked with two different embodiments of medical devices of the present invention. In particular, a tubular medical device 160 (as depicted in FIG. 7) may be used in one leak while a low profile medical device 130 (similar to that depicted in FIGS. 4A and 4B) with a frame member 132 configured to support, for example, a foam curtain as a tissue in-growth member 134, disposed around a curved edge of the artificial valve 100. Thus, multiple device may be employed depending, for example, on the type and size of valvular leak that needs to be blocked or occluded.

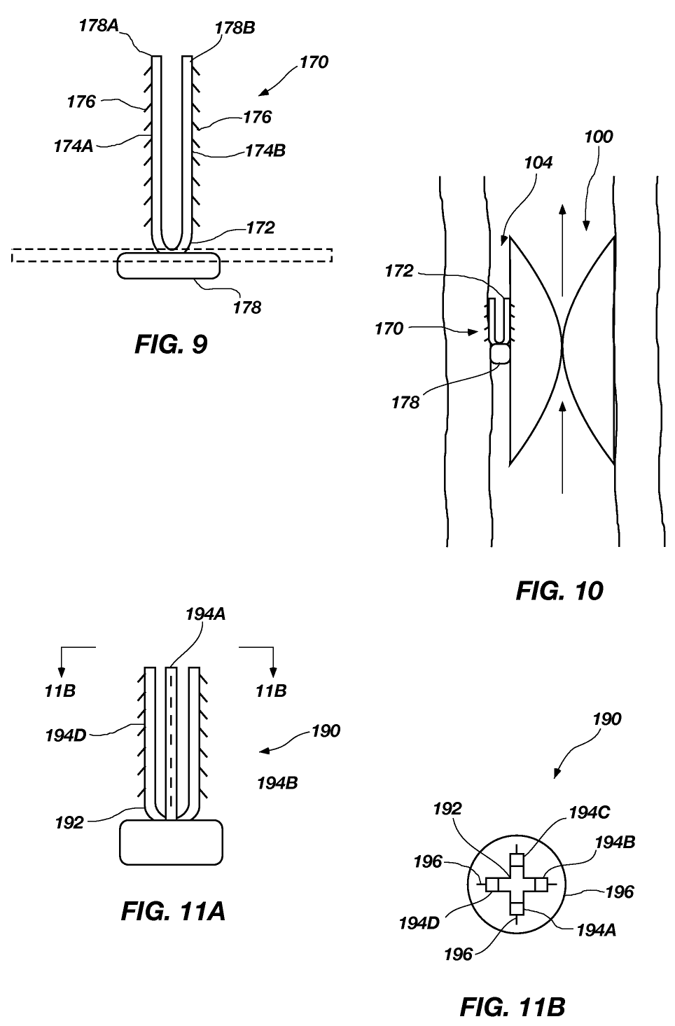

FIG. 9 illustrates a medical device 170 according to yet another embodiment of the present invention. The medical device 170 includes a frame member 172 having a plurality of arms 174A and 174B formed from, for example, wire or strip. The arms 174A and 174B may also be formed by cutting them from sheet or tubing with any appropriate method such as, for example, laser cutting.

Tines 176 may be formed on, or otherwise associated with, the arms 174A and 174B and configured to grab or engage the tissue wall and prevent migration of the medical device 170 from the valvular leak. The arms 174A and 174B may be constructed of a material with elastic memory such as shape memory alloy including, for example Nitinol. The arms 174A and 174B may have an unconstrained shape that is naturally “open” as shown by the substantially horizontally extending dashed lines in FIG. 9. It should be noted that the arms 174A and 174B can be configured to naturally “open” at any desired angle, such as 60 degrees, so as to provide the desired force in the arms to sufficiently anchor the tines 176. The arms 174A and 174B may be compressed into a “delivery” shape where the tips 178A and 178B are displaced towards one another as generally shown in FIG. 11. The medical device 170 may be introduced into the valvular leak in the compressed delivery shape and then released within the space defining the leak. The elastic memory of the arms 174A and 174B causes the arms 174A and 174B to move toward the “open” shape until it engages and presses against the sides of the opening forming the valvular leak with the tines 176 engaging tissue of the leak opening as shown in FIG. 10.

The medical device 170 may also include a tissue in-growth member 178 at various locations on the frame member 172. As with other embodiments described herein, the tissue in-growth member 178 may be constructed of polymer fibers such as Dacron or PTFE fibers, polymer foam such as polyester foam, polymer fabric, a combination of these or any other suitable material configured to encourage or promote tissue growth. The tissue in-growth members 178 may exhibit a variety of different shapes or configurations. For example, the tissue in-growth members 178 may be shaped substantially like a ball or sphere, they may be crescent shaped, or the material may wrapped along or around the frame member 172. Other shapes and configuration that provide a sufficient plug for the valvular leak are also contemplated.

Referring briefly to FIGS. 11A and 11B, a medical device 190 is shown that is generally similar to the embodiment described above with respect to FIG. 9. The medical device 190 includes a frame member 192 having a plurality of arms 194A-194D and a tissue in-growth member 196. Tines 198 may be associated with one or more of the arms 194A-194D for engaging and grabbing surfaces surrounding the valvular leak. As compared to the medical device 170 shown in FIG. 9 (which includes two arms 174A and 174B extending away from each other in a single plane), the medical device 190 includes multiple arms extending in more than one plane. Thus, depending on the configuration of the valvular leak that needs to be plugged, a multi-plane device may be more effective than a single plane device as has been demonstrated hereinabove with respect to other embodiments.

Other medical devices may also be utilized to effect closure or blocking of valvular leak. For example, some of the devices set forth in U.S. patent application Ser. No. 12/253,831, filed on Oct. 17, 2008, entitled MEDICAL DEVICE FOR MODIFICATION OF LEFT ATRIAL APPENDAGE AND RELATED SYSTEMS AND METHODS, (the disclosure of which is incorporated herein in its entirety), may be utilized to block or close a valvular leak. For example, an expandable foam body might be utilized to block or occlude a valvular leak. While the specific examples of expandable foam members shown in the referenced co-pending application depict a substantially spherical member as one example, other shapes may be utilized including oval, crescent or generally “flattened” geometries.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

We claim:

1. A medical device for repairing a paravalvular leak, comprising:
at least one multicellular frame member configured to be implanted at a paravalvular leak; and
at least one tissue in-growth member associated with the frame member, the tissue in-growth member configured to promote tissue growth and permanently maintain the frame member at the leak.

2. The medical device of claim 1, wherein the at least one multicellular frame member is a substantially tubular structure.

3. The medical device of claim 1, wherein the at least one tissue in-growth member is disposed within an internal space defined by the substantially tubular structure.

4. The medical device of claim 3, wherein the at least one multicellular frame member is formed of a material comprising a shape memory alloy.

5. The medical device of claim 4, wherein the shape memory alloy comprises a nickel-titanium alloy.

6. The medical device of claim 1, wherein the at least one tissue in-growth material is formed of a polymer material.

7. The medical device of claim 1, wherein the at least one multicellular frame member is a substantially planar structure.

8. The medical device of claim 7, wherein the at least one multicellular frame member is formed of a material comprising a shape-memory alloy.

9. The medical device of claim 1, further comprising a plurality of tines associated with the at least one multicellular frame member, the plurality of tines being configured to engage a wall of a valvular leak and anchor the medical device relative thereto.

10. The medical device of claim 1, wherein the at least one multi-cellular frame member comprises a first frame member and a second frame member, and wherein the at least one tissue in-growth member is disposed between the first frame member and the second frame member.

11. The medical device of claim 10, wherein the first frame member and the second frame member are substantially planar members and are arranged to lie in substantially parallel planes.

12. A method of closing a paravalvular leak, the method comprising:
disposing a multicellular frame member within the paravalvular leak; and
disposing a tissue in-growth member within the paravalvular leak at a location adjacent the multicellular frame member to promote tissue growth and permanently maintain the frame member at the leak.

13. The method according to claim 12, wherein disposing a multicellular frame member within the paravalvular leak and disposing a tissue in-growth member within the paravalvular leak occurs substantially simultaneously.

14. The method according to claim 12, wherein disposing a multicellular structure includes disposing a self expanding structure within the paravalvular leak.

15. The method according to claim 12, wherein disposing a self expanding structure includes disposing a substantially planar structure within the paravalvular leak.

16. The method according to claim 12, wherein disposing a self expanding structure includes disposing a substantially tubular structure within the paravalvular leak.

17. The method according to claim 14, wherein disposing a tissue in-growth member includes disposing the tissue in-growth member within an inner space defined by the substantially tubular structure.

18. The method according to claim 15, wherein disposing a tissue in-growth member within the paravalvular leak further comprises disposing a foam material within the paravalvular leak.

19. The method according to claim 12, wherein disposing a multicellular frame member within the paravalvular leak and disposing a tissue in-growth member within the paravalvular leak further comprises disposing a first frame member and a second frame member, with the tissue in-growth member disposed between the first and second frame members, within the paravalvular leak.

20. The method according to claim 19, further comprising configuring the first and second frame members to be substantially planar members and arranging the first and second frame members to lie in substantially parallel planes.

21. A medical device for repairing a paravalvular leak, comprising:
a frame member having at least two elongated arms biased in two different directions; and
a tissue in-growth member configured to be attached to the frame member to promote tissue growth and permanently seal the leak.

22. The medical device of claim 21, wherein the at least two different directions include a first direction and a second direction that is substantially opposite of the first direction.

23. The medical device of claim 21, wherein the at least two elongate arms include a first pair of elongated arms and a second pair of elongated arms.

24. The medical device of claim 21, wherein the frame member is formed of a material comprising a shape-memory alloy.

25. The medical device of claim 24, wherein the shape-memory alloy comprises a nickel-titanium alloy.

26. The medical device of claim 24, wherein the tissue in-growth member is formed of a polymer material.
